(19) United States
(12) Patent Application Publication
(10) Pub. No.: US 2023/0148988 A1
(43) Pub. Date: May 18, 2023

(54) DIGITAL STETHOSCOPE DEVICE AND ITS WORKING METHOD THEREOF

(71) Applicants: Mohsin Ali Khan, Lucknow (IN); Zaw Ali Khan, Lucknow (IN); Sarina Zehra, Lucknow (IN); Kinza Zehra, Lucknow (IN); Eras Lucknow Medical College & Hospital, Lucknow (IN); American University of Barbados, Bridgetown (BB)

(72) Inventors: Mohsin Ali Khan, Lucknow (IN); Zaw Ali Khan, Lucknow (IN); Sarina Zehra, Lucknow (IN); Kinza Zehra, Lucknow (IN)

(21) Appl. No.: 18/157,348

(22) Filed: Jan. 20, 2023

(51) Int. Cl. A61B 7/04; A61B 7/00

(52) U.S. Cl. CPC A61B 7/04; A61B 7/003

(57) ABSTRACT

The present invention generally relates to a digital stethoscope device comprises a chest piece coupled to a diaphragm to collect the vibrations with the help of a sensing node; an analog-to-digital converter to convert the sound signals from the heart to analog electrical signals; a pre-amplifier with a small gain to suppress the interference from power lines and further transmit the signals to power amplifier; an anti-aliasing filter to prevent aliasing effect; and a display unit for receiving and displaying the output signals via a wireless personal area network.

### FIELD OF THE INVENTION

The present disclosure relates to a digital stethoscope device and its working method. In more detail, the device is used for monitoring and listening heart and lung sounds of patient in real time using communication means and applications even if they are away from the patient.

### BACKGROUND OF THE INVENTION

An electronic stethoscope having a vibration transducer, a speaker, an earphone plan, and something like one computerized channel for laying out no less than one motivation move capability relating to no less than one acoustic stethoscope type.

In this manner, the signs heard will relate to those learned, and subsequently the upsides of more prominent enhancement and disposal of clamor sources might be completely used. The capacity to make up for a singular specialist's hearing misfortune is empowered.

Moreover, with stereoscopy exemplifications, sound recurrence conveyances can be changed to spatial or worldly sound appropriations discernible by the client working with, for instance, the location of heart mumbles and the taking of circulatory strain.

In the view of the forgoing discussion, it is clearly portrayed that there is a need to have a digital stethoscope device and its working method.

### SUMMARY OF THE INVENTION

The present disclosure seeks to provide a digital stethoscope device and method for listening (auscultation) and interpretation of the sounds of heart and lungs. The device enables doctors and healthcare workers to monitor and listen heart and lung sounds of patient in real time using communication means and applications even if they are away from the patient wherein said stethoscope comprises of chest piece with diaphragm to collect the vibrations with the help of microphone or piezoelectric sensors the sound signals from the heart are converted to analog electrical signals by the analog-to-digital converter; a pre-amplifier with a small gain is used to suppress the interference from power lines; further pre amplifier transmits these signals to power amplifier; an anti-aliasing filter is then employed to prevent aliasing effect.

In an embodiment, a digital stethoscope device is disclosed. The device includes a chest piece coupled to a diaphragm to collect the vibrations with the help of a sensing node. The device further includes an analog-to-digital converter to convert the sound signals from the heart to analog electrical signals. The device further includes a pre-amplifier with a small gain to suppress the interference from power lines and further transmit the signals to power amplifier. The device further includes an anti-aliasing filter to prevent aliasing effect. The device further includes a display unit for receiving and displaying the output signals via a wireless personal area network.

An object of the present disclosure is to monitor and listen heart and lung sounds of patient in real time using communication means and applications even if they are away from the patient.

Another object of the present disclosure is to promote multi-inspection at once using multiple headphones.

Yet another object of the present invention is to deliver an expeditious and cost-effective digital stethoscope device.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a digital stethoscope device in accordance with an embodiment of the present disclosure;

FIG. 2 illustrates a flow chart of a working method for digital stethoscope device in accordance with an embodiment of the present disclosure;

FIG. 3 illustrates a schematic diagram of a digital stethoscope device of in accordance with an embodiment of the present disclosure; and

FIG. 4 illustrates a connection diagram of multi-inspection at once using multiple headphones in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

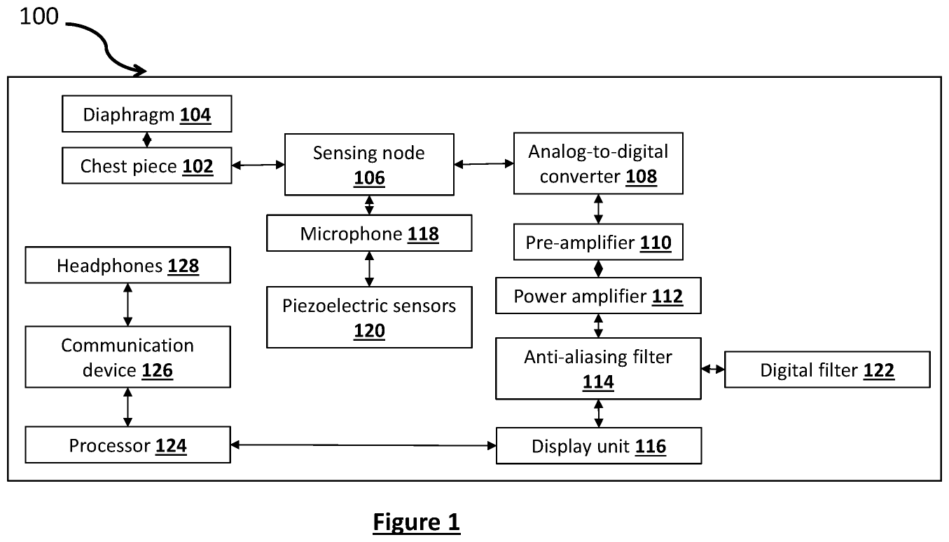

Referring to FIG. 1, a block diagram of a digital stethoscope device is illustrated in accordance with an embodiment of the present disclosure. The device 100 includes a chest piece 102 coupled to a diaphragm 104 to collect the vibrations with the help of a sensing node 106.

In an embodiment, an analog-to-digital converter 108 is used to convert the sound signals from the heart to analog electrical signals.

In an embodiment, a pre-amplifier 110 is used with a small gain to suppress the interference from power lines and further transmit the signals to power amplifier 112.

In an embodiment, an anti-aliasing filter 114 is used to prevent aliasing effect.

In an embodiment, a display unit 116 is used for receiving and displaying the output signals via a wireless personal area network.

In another embodiment, the sensing node 106 is preferably selected from a microphone 118 and piezoelectric sensors 120.

In another embodiment, the filter section is built with a band pass filter circuit having the frequency range of most HS signals.

In another embodiment, a digital filter 122 is used to extract the signal within the frequency band of interest from the noisy data.

In another embodiment, an advanced artifacts removal is used for output signal-to-noise ratio improvement.

In another embodiment, the processor 124 carries out the Signal processing to convert the raw data into presentable data for further analysis.

In another embodiment, device is connected to any application through a communication device 126 preferably selected from a Bluetooth and with the help of the same the practitioner is able to listen, monitor and even record heart and lungs sound remotely.

In another embodiment, a plurality of headphones 128 is connected with device to listen live heart and lungs sound remotely by a plurality of practitioners.

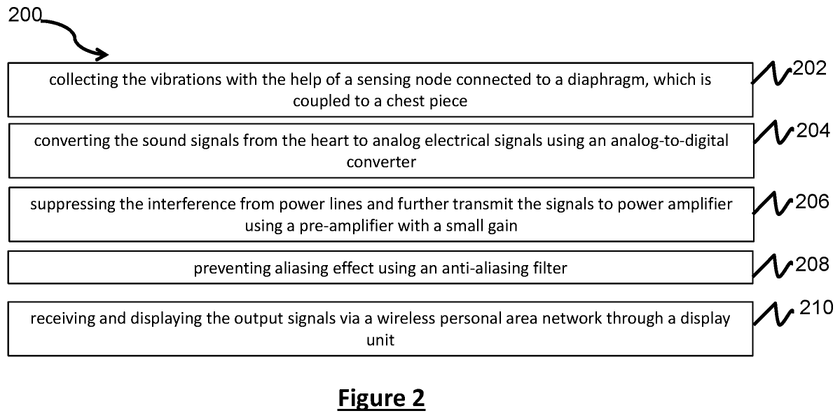

FIG. 2 illustrates a flow chart of a working method for a digital stethoscope device in accordance with an embodiment of the present disclosure. At step 202, the method 200 includes collecting the vibrations with the help of a sensing node 104 connected to a diaphragm 102, which is coupled to a chest piece.

At step 204, the method 200 includes converting the sound signals from the heart to analog electrical signals using an analog-to-digital converter 106.

At step 206, the method 200 includes suppressing the interference from power lines and further transmit the signals to power amplifier 110 using a pre-amplifier with a small gain.

At step 208, the method 200 includes preventing aliasing effect using an anti-aliasing filter 112.

At step 210, the method 200 includes receiving and displaying the output signals via a wireless personal area network through a display unit 114.

In another embodiment, sensing vibrations using the sensing node 104 preferably selected from a microphone 116 and piezoelectric sensors 118.

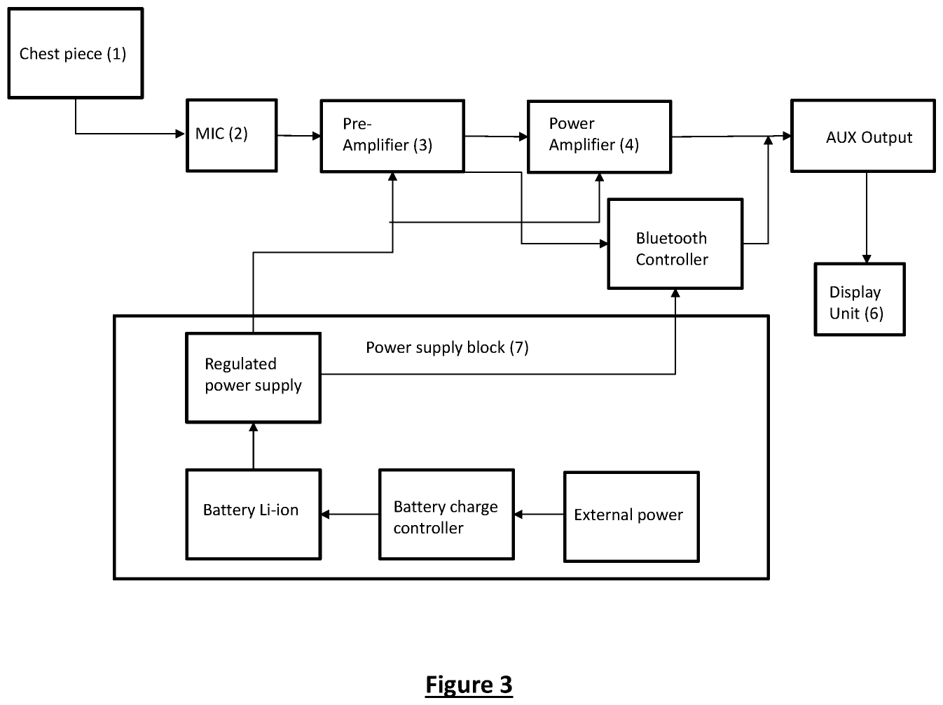

FIG. 3 illustrates a schematic diagram of a digital stethoscope device of in accordance with an embodiment of the present disclosure.

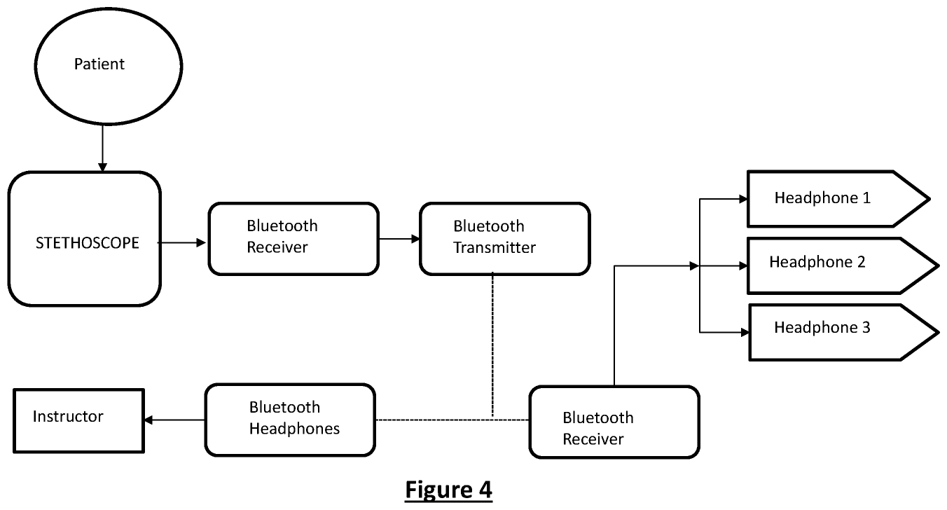

FIG. 4 illustrates a connection diagram of multi-inspection at once using multiple headphones in accordance with an embodiment of the present disclosure.

The concept behind multiple headphones connected with our stethoscope is that It helps the students to listen to live heart and lungs sound when their instructor places the stethoscope on patients chest. Students and their instructor both listen to the sound simultaneously. The instructor will explain the sound so students can understand better. It's easy to understand, and helps in live training, and useful in decision taking.

The patient wherein said stethoscope comprises of chest piece with diaphragm to collect the vibrations with the help of microphone or piezoelectric sensors; the sound signals from the heart are converted to analog electrical signals by the analog-to-digital converter; a pre-amplifier with a small gain is used to suppress the interference from power lines; further pre amplifier transmits these signals to power amplifier; an anti-aliasing filter is then employed to prevent aliasing effect.

A Stethoscope for listening (auscultation) and interpretation of the sounds of heart and lungs as claimed in any of the claims wherein the device can be connected to any application and with the help of the same the practitioner can listen, monitor and even record heart and lungs sound remotely.

The concept behind multiple headphones connected with our stethoscope is that it helps the students to listen to live heart and lungs sound when their professor places the stethoscope on patient's chest and students and their professor both listen to the sound simultaneously and the professor will explain the sound so students can understand better.

Stethoscopes are utilized by doctors to pay attention to sounds from the life form, specifically heart and lungs. The peculiarities tuned in for produce sounds with frequencies from under 16 Hz to around 8 kHz, yet a serious low pass sifting happens during the entry of tissue and skin. The skin behaves like a transmitter of those signs which are hence open.

The development of the stethoscope discovers that main a little region of the skin is paid attention to at a time, and that sounds in the room outside are hosed, and in this way, the sign to-clamor proportion is to some degree gotten to the next level. Doctors train effectively in the utilization of stethoscopes, and subsequently their capacity to recognize signals in the encompassing commotion might ascend by around 15 dB.

A stethoscope is a widespread device. In any case, it's worth or potentially execution to the clients has fallen, because of the specialized advancement of society. The expanded machine clamor, specifically in emergency clinics, by and by, makes the signs lie underneath, or at the most at, the lower furthest reaches of human hearing.

To this is added that increasingly more youngster's experience the ill effects of hearing misfortune at the time they might leave on a clinical training, and subsequently the acoustic stethoscope has by and by arrived at its constraint of execution.

The device is a Stethoscope for listening (auscultation) and interpretation of the sounds of heart and lungs, which enables doctors and healthcare workers to monitor and listen heart and lung sounds of patient in real time using communication means and applications even if they are away from the patient wherein said stethoscope comprises of chest piece with diaphragm to collect the vibrations with the help of microphone or piezoelectric sensors; the sound signals from the heart are converted to analog electrical signals by the analog-to-digital converter; a pre-amplifier with a small gain is used to suppress the interference from power lines; further pre amplifier transmits these signals to power amplifier; an anti-aliasing filter is then employed to prevent aliasing effect.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A digital stethoscope device, the device comprises:
a chest piece coupled to a diaphragm to collect the vibrations with the help of a sensing node;
an analog-to-digital converter to convert the sound signals from the heart to analog electrical signals;
a pre-amplifier with a small gain to suppress the interference from power lines and further transmit the signals to power amplifier;
an anti-aliasing filter to prevent aliasing effect; and
a display unit for receiving and displaying the output signals via a wireless personal area network.

2. The device as claimed in claim 1, wherein the sensing node is preferably selected from a microphone and piezoelectric sensors.

3. The device as claimed in claim 1, wherein the filter section is built with a band pass filter circuit having the frequency range of most HS signals.

4. The device as claimed in claim 1, wherein a digital filter is used to extract the signal within the frequency band of interest from the noisy data.

5. The device as claimed in claim 1, further comprises an advanced artifacts removal is used for output signal-to-noise ratio improvement.

6. The device as claimed in claim 1, wherein the processor carries out the signal processing to convert the raw data into presentable data for further analysis.

7. The device as claimed in claim 1, wherein device is connected to any application through a communication device preferably selected from a Bluetooth and with the help of the same the practitioner is able to listen, monitor and even record heart and lungs sound remotely.

8. The device as claimed in claim 1, wherein a plurality of headphones is connected with device to listen live heart and lungs sound remotely by a plurality of practitioners.

9. A working method for digital stethoscope device, the method comprises:
collecting the vibrations with the help of a sensing node connected to a diaphragm, which is coupled to a chest piece;
converting the sound signals from the heart to analog electrical signals using an analog-to-digital converter;
suppressing the interference from power lines and further transmit the signals to power amplifier using a pre-amplifier with a small gain;
preventing aliasing effect using an anti-aliasing filter; and
receiving and displaying the output signals via a wireless personal area network through a display unit.

10. The method as claimed in claim 9, wherein sensing vibrations using the sensing node preferably selected from a microphone and piezoelectric sensors.
